(19) United States
(12) Patent Application Publication
(10) Pub. No.: US 2026/0218262 A1
(43) Pub. Date: Jul. 30, 2026

(54) SCARLESS TEMPLATE-FREE ENZYMATIC SYNTHESIS OF POLYNUCLEOTIDES

(71) Applicant: DNA SCRIPT, Le Kremlin-Bicêtre (FR)

(72) Inventors: Adrian HORGAN, Le Kremlin-Bicêtre (FR); Damiano VERARDO, Le Kremlin-Bicêtre (FR); Beatrice ADELIZZI, Le Kremlin-Bicêtre (FR); Daniel RODRIGUEZ-PINZON, Le Kremlin-Bicêtre (FR); Emma THOMEE, Le Kremlin-Bicêtre (FR)

(21) Appl. No.: 19/148,244

(22) PCT Filed: Jan. 16, 2024
(86) PCT No.: PCT/EP2024/050931

§ 371 Date: Jul. 15, 2025

(30) Foreign Application Priority Data
Jan. 16, 2023 (EP) 23315007.7

(51) Int. Cl. C12P 19/34

(52) U.S. Cl. CPC C12P 19/34

(57) ABSTRACT

The present invention is directed to methods and kits for template-free enzymatic synthesis of polynucleotides using chain elongation conditions that suppress the formation of DNA secondary structures including, but not limited to, intra-strand and between-strand duplexes, G-quadruplexes, and the like. In some embodiments, such chain elongation conditions include using 3′-O-blocked dNTP monomers that base protection groups or base analogs that suppress the formation of hydrogen bonding in the polynucleotide being synthesized.

### BACKGROUND OF THE INVENTION

Interest in enzymatic approaches to polynucleotide synthesis has recently increased both because of increased demand for synthetic polynucleotides in many areas, such as synthetic biology, CRISPR-Cas9 applications, high-throughput sequencing, and the like, and because of the limitations of chemical approaches to polynucleotide synthesis (Jensen et al. (2018) Biochemistry 57:1821-1832). Currently, most enzymatic approaches employ a template-free polymerase to repeatedly add 3′-O-protected nucleoside triphosphates to an initiator or an elongated strand attached to a support followed by deprotection until a polynucleotide of the desired sequence is obtained. Among the challenges of devising a practical implementation of such enzymatic synthesis is to find a cost-effective and efficient way to cleave a desired polynucleotide product from the initiator sequence and the support.

Cleavage of DNA on planar supports has achieved only modest levels of DNA release. Although not wishing to be bound by theory, the DNA synthesized on a planar support is often at high enough density to sterically block access of cleavage enzymes to a DNA cleavage site, which is generally placed close to the end of the DNA that is attached to the surface of the planar support. The longer the DNA strand, the more the enzyme is sterically blocked from reaching the cleavage site. Reducing DNA density on the planar support is not desirable for applications where parallelization and high-throughput production are important. Non-enzymatic means of DNA cleavage with small molecule cleavage agents or photocleavage with light generally leave a DNA scar. Thus, there remains a need for more efficient and scarless methods of cleaving polynucleotides from a support after enzymatic synthesis.

### SUMMARY OF THE INVENTION

Methods are provided for template-free enzymatic synthesis of polynucleotides on a solid support. For enzymatic synthesis, an initiator is used, typically an oligonucleotide or polynucleotide at least three nucleotides in length, comprising a free 3′-hydroxyl group, which is extended using a template-free polymerase. The subject methods use an initiator that further comprises i) a cleavable group, which can be cleaved by a small molecule chemical agent, adjusting pH, heat, or photocleaved by illumination with light to release polynucleotides from a solid support after enzymatic synthesis is completed, and ii) an enzymatic cleavage site, which can be cleaved by a cleavage enzyme to remove scars from the polynucleotides.

In one aspect, a method of enzymatically synthesizing a plurality of polynucleotides is provided, the method comprising: (a) providing a plurality of reaction sites on a solid support; (b) immobilizing a plurality of initiators on the surface of the solid support, wherein each initiator comprises a free 3′-hydroxyl group, at least one cleavable group, and at least one enzymatic cleavage site, wherein said at least one enzymatic cleavage site is positioned between the free 3′-hydroxyl group and said at least one cleavable group; (c) contacting the surface with a 3′-O-protected nucleoside triphosphate and a template-free polymerase under suitable conditions for enzymatic extension of the initiators, wherein incorporation of the 3′-O-protected nucleoside triphosphate results in production of a 3′-O-protected extension product at each of the reaction sites; (d) performing one or more cycles of i) deprotecting the 3′-O-protected extension products at the reaction sites, wherein extension products having free 3′-hydroxyl groups are formed at the reaction sites, and ii) contacting the surface with another 3′-O-protected nucleoside triphosphate and a template-free polymerase under suitable conditions for enzymatic extension of the extension products having free 3′-hydroxyl groups; (e) repeating step (d) until synthesis of the plurality of polynucleotides is completed; (f) cleaving said at least one cleavable group, thereby releasing the plurality of polynucleotides from the surface; and (g) enzymatically cleaving said at least one enzymatic cleavage site, thereby removing polynucleotide scars from the plurality of polynucleotides to produce a plurality of scarless polynucleotides, wherein said enzymatically cleaving the at least one enzymatic cleavage site is performed simultaneously with or after said cleaving the at least one cleavable group.

In certain embodiments, the solid support is a bead. In other embodiments, the solid support is planar.

In certain embodiments, the plurality of reaction sites is organized in a spatially addressable array on the solid support.

In certain embodiments, the method further comprises washing the surface after a round of enzymatic extension to remove the polymerase and free nucleotides that are not incorporated into the extension products before performing a subsequent round of enzymatic extension.

In certain embodiments, the template-free polymerase is selected from the group consisting of a terminal deoxynucleotidyl transferase (TdT), a translesion DNA polymerase of type η, a translesion DNA polymerase of type ζ, a polynucleotide phosphorylase (PNPase), a template-independent RNA polymerase, a terminal transferase, a template-independent DNA polymerase, a reverse transcriptase, and a 9° N DNA polymerase, wherein the enzymatic cleavage sites and the cleavable groups do not interfere with enzymatic extension of the initiators.

In certain embodiments, each initiator comprises an oligonucleotide comprising at least 3 nucleotides in length.

### BRIEF DESCRIPTION OF THE DRAWINGS

FIG. 1. A schematic showing photochemical release of DNA and enzymatic cleavage to remove a DNA scar.

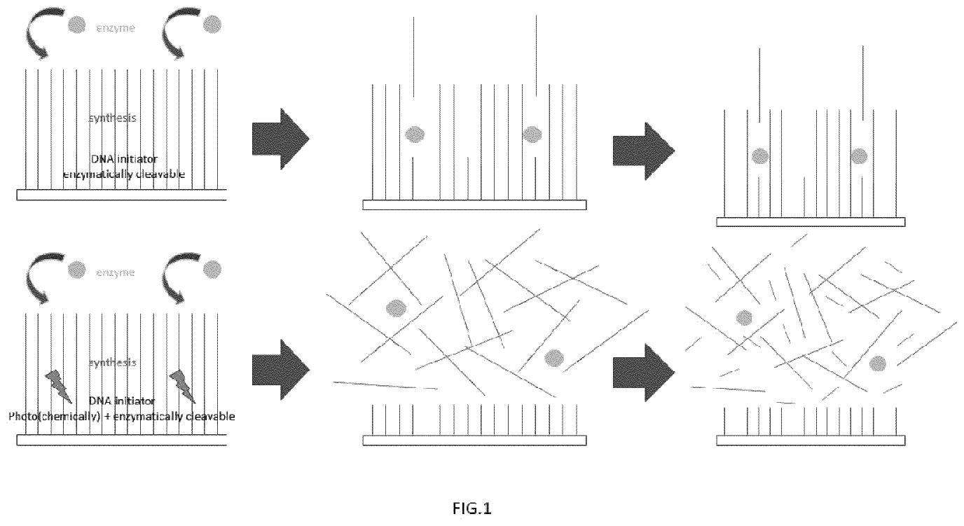

FIG. 2. Design of DNA/RNA initiator. The DNA/RNA initiator preferably has catchable groups on the part that is released into solution and generates a scar. The enzyme also preferably has catchable groups.

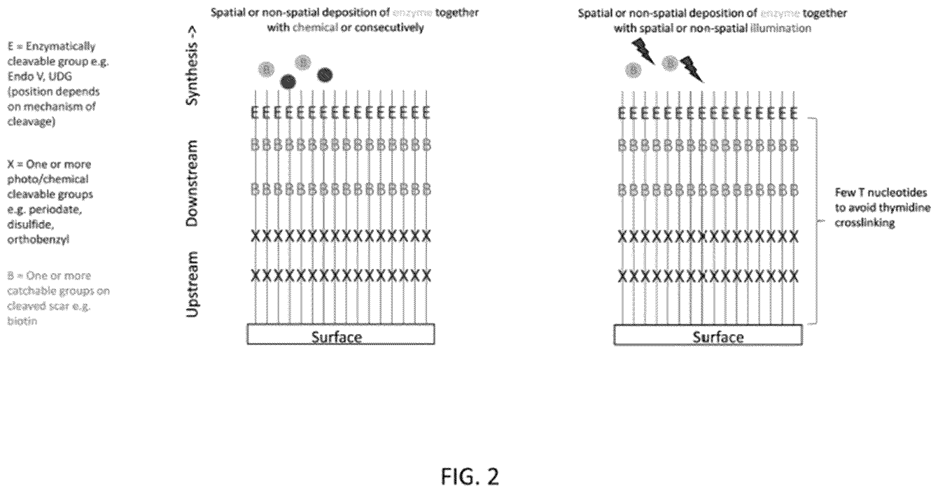

FIG. 3. Problem of photocrosslinking of thymidine bases in DNA. Illuminating DNA with polyT sequences should be avoided to prevent photocrosslinking of thymidine bases. An RNA initiator may be used instead of a DNA initiator to avoid photocrosslinking of thymidine bases because RNA has better UV resistance than DNA.

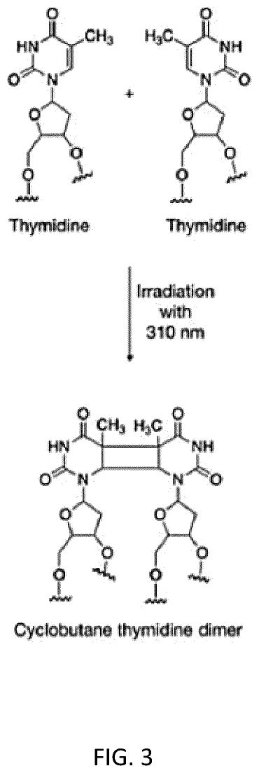

FIG. 4. Summary of cleaving yields with a single cleavable group, two cleavable groups, and multiple cleavable groups. We predicted that multiple groups of the same type would give higher surface cleavage yields from surfaces. The improvement would depend on how efficient a single cleavable group of a particular type is. If a given type is highly efficient (alkaline cleavage of rU), there will be proportionally less benefit of having 2 groups of this type and even less of having 3 groups. The best yields of the scarless oligo product were obtained using a double cleavage strategy. Preferably, the 2 cleavable groups are different and the group closest to the 3′ end is an enzyme cleavable group (e.g., dI or dU).

FIG. 5. Percentage of total substitution rate when photocleaving is performed under ambient conditions (experiments labelled NGS1 and NGS2, two repeats) and under argon flow (experiments labelled NGS3 and NGS4, two repeats), both for the e13 sequence (FIG. 5A) and for the cs sequence (FIG. 5B).

FIG. 6. Details of the effect of argon gas flow (right column) on a substitution type by substitution type basis, both for the cs sequence (above) and the e13 sequence (below), by comparison to ambient conditions (left column)

### DETAILED DESCRIPTION

Methods are provided for template-free enzymatic synthesis of polynucleotides on a solid support using double cleavage to produce scarless polynucleotides. In particular, methods are disclosed that use an initiator that comprises a cleavable group, to release polynucleotides from a solid support after enzymatic synthesis is completed, and an enzymatic cleavage site, which can be cleaved by a cleavage enzyme to remove polynucleotide scars.

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a polynucleotide” includes a plurality of such polynucleotides, and reference to “the initiator” includes reference to one or more initiators and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “polynucleotide” “oligonucleotide”, “nucleic acid”, and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide”, “oligonucleotide”, “nucleic acid”, and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide”, “oligonucleotide”, “nucleic acid”, and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), bases containing linking groups permitting the attachment of labels, such as fluorophores, or haptens, and the like, as well as unmodified forms of the polynucleotide or oligonucleotide. In particular, DNA is deoxyribonucleic acid. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Whenever the use of an oligonucleotide, polynucleotide, or nucleic acid requires enzymatic processing, such as extension by a polymerase, ligation by a ligase, or the like, one of ordinary skill would understand that oligonucleotides or polynucleotides in those instances would not contain certain analogs of intenucleosidic linkages, sugar moieties, or bases at any or some positions. Polynucleotides typically range in size from a few monomeric units, e.g., 5-40, when they are usually referred to as “oligonucleotides,” to several thousand monomeric units. Whenever a polynucleotide, oligonucleotide, or nucleic acid is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually, polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

The terms “base protecting moiety” and “base protecting group” are used interchangeably and refer to a protecting group on a nucleotide base, which may be used to reduce or eliminate the formation of secondary structure in the course of polynucleotide chain extensions and/or prevent deamination (see, e.g., International Patent Application Publication No. WO 2021/018921, herein incorporated by reference in its entirety). A base protecting group may be attached to the 6-nitrogen of deoxyadenosine triphosphate, the 2-nitrogen of deoxyguanosine triphosphate, and/or the 4-nitrogen of deoxycytidine triphosphate. In some embodiments, a base protecting group is attached to all of the indicated nitrogens. In some embodiments, a base protecting group attached to a 6-nitrogen of deoxyadenosine triphosphate is selected from the group consisting of benzoyl, phthaloyl, phenoxyacetyl, and methoxy acetyl; a base protecting group attached to the 2-nitrogen of deoxyguanosine triphosphate is selected from the group consisting of isobutyryl, isobutyryloxyethylene, acetyl, 4-isopropyl-phenoxyacetyl, phenoxyacetyl, and methoxyacetyl; and a base protecting group attached to said 4-nitrogen of deoxycytidine triphosphate is selected from the group consisting of benzoyl, phthaloyl, acetyl, and isobutyryl. In some embodiments, a protecting group attached to the 6-nitrogen of deoxyadenosine triphosphate is benzoyl; a base protecting group attached to the 2-nitrogen of deoxyguanosine triphosphate is isobutryl or dimethylformamidine; and the base protecting group attached to the 4-nitrogen of deoxycytidine triphosphate is acetyl. In some embodiments, a base protecting group attached to the 6-nitrogen of deoxyadenosine triphosphate is phenoxyacetyl; a base protecting group attached to the 2-nitrogen of deoxyguanosine triphosphate is 4-isopropyl-phenoxyacetyl or dimethylformamidine; and the base protecting group attached to the 4-nitrogen of deoxycytidine triphosphate is acetyl. In some embodiments, base protecting moieties are removed (i.e. the product is deprotected) and product is cleaved from a solid support in the same reaction. For example, an initiator may comprise a ribo-uridine which may be cleaved to release the polynucleotide product by treatment with 1 M KOH, or like reagent (ammonia, ammonium hydroxide, NaOH, or the like), which simultaneously removes base-labile base protecting moieties.

As used herein, the term “initiator”, “DNA initiator”, “initiating fragment”, “initiator nucleic acid”, “initiator oligonucleotide”, or “initiator polynucleotide” refers to an oligonucleotide or polynucleotide comprising a free 3′-hydroxyl group, which can be further elongated by a template-free polymerase (e.g., TdT). In some cases, the initiator further comprises a cleavable group and an enzymatic cleavage site.

As used herein, the term “extension product”, “extension intermediate”, “elongation product” or “elongation intermediate” are used interchangeably and refer to the product resulting from enzymatic extension at the 3′ end of an initiator or of an extension intermediate generated from an initiator by a template-free polymerase.

As used herein, the term “scarless” refers to polynucleotides containing a desired sequence of interest with no extraneous sequences. In specific embodiments, “scarless” refers to polynucleotides containing a desired sequence of interest with no extraneous sequences, said polynucleotides also having an intact 5′ phosphate.

As used herein, the term “cleavable group” refers to a group which can be cleaved by a small molecule chemical agent, by adjusting pH, by heat, or which can be photocleaved by illumination with light to release polynucleotides from a solid support after enzymatic synthesis is completed. In particular embodiments, the cleavable group can be cleaved by an enzyme, such as Rnase A (to cleave the group rU) or Endo V (to cleave the group dl). In other embodiments, the cleavable group does not include groups cleavable by enzymatic means.

A solid support is “addressable” when it has multiple features (e.g., electrodes, reaction centers) positioned at particular predetermined locations (e.g., “addresses”) on the surface of the solid support.

An “array” includes any two-dimensional or substantially two-dimensional (as well as a three-dimensional) arrangement of addressable regions, e.g., spatially addressable regions. An array is “addressable” when it has multiple features (e.g., electrodes, reaction centers) positioned at particular predetermined locations (e.g., “addresses”) on the array. Array features may be separated by intervening spaces.

As used herein, “inkjet assisted synthesis” means that one or more synthesis reagents are delivered to reaction sites in droplets generated by one or more inkjet pumps.

“Synthesis reagents” include any reagent used in a synthesis cycle to couple a monomer, particularly a 3′-O-protected-nucleoside triphosphate, to an initiator or elongated fragment, such as, a template-free polymerase, 3′-O-protected nucleoside triphosphates, divalent cations or other cofactors, deprotection (or deblocking) reagents, and the like.

The terms “deprotection” agent, buffer, solution, or the like, are used synonymously herein to the terms “deblocking” agent, buffer, solution, or the like, respectively. Likewise, the term “protected” in reference to compounds, such as, dNTPs, is used synonymously with the term “blocked” in reference to compounds.

As used herein, the term “deprotection solution” (or its equivalent terms) means a reagent that brings about or promotes the removal of a protection group, for example, a 3′-O-protecting group of a nucleotide. As described more fully below, the composition of a deprotection solution (and deprotection reaction conditions) depends on the nature of the protecting group (or blocking group) which is to be removed. In various embodiments, a deprotection solution may contain specific reagents that chemically react with a protection group and/or protected moiety (such as, a reducing agent like TCEP (tris(2-carboxyethyl)phosphine)), enzymes for enzymatic cleavage, scavengers, co-factors, or the like. In some embodiments, a deprotection solution may not contain specific reagents that react with a protection group, but may contain components, e.g. pH buffers, that are compatible with or promote physical cleavage of a protecting group, such as in the case of a photocleavable protecting group. Typically, in a reaction cycle for elongating a polynucleotide fragment, in a deprotecting step a deprotection solution is incubated with 3′-O-protected elongated fragments for a predetermined incubation time. Typical incubation times (i.e. durations of incubating steps) are in the range of from 1 minute to 30 minutes; or in the range of from 3 minutes to 30 minutes; or in the range of from 3 minutes to 15 minutes. Typical elongation reaction temperatures are in the range of from room temperature (RT) to 80° C.; or from 20° C. to 80° C.; or from 20° C. to 60° C.

“Synthesis reagents” also include reagents for preparing a substrate for polynucleotide synthesis, such as, reagents for defining reaction sites, initiators, capping reagents, and the like.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

As used herein, the term “capture agent” refers to a molecule that can selectively bind and capture a catchable group, i.e., a moiety the capture agent selectively binds to on a target molecule (e.g., polynucleotide scar, cleavage enzyme). By “capture” is meant that the target molecule comprising the catchable group can be separated from other components of a sample by virtue of the binding of the capture molecule to the catchable group. Typically, the capture molecule is associated with a solid support, either directly or indirectly.

As used herein, the term “capture oligonucleotide” refers to an oligonucleotide that contains a nucleotide sequence complementary to a nucleotide sequence present in a target nucleic acid analyte such that the capture oligonucleotide can “capture” the target nucleic acid. The polynucleotide regions of a capture oligonucleotide may be composed of DNA, and/or RNA, and/or synthetic nucleotide analogs.

As used herein, the term “catchable group” refers to a moiety that a capture agent selectively binds to.

A “restriction endonuclease” or “restriction enzyme” is a member of a family of enzymes that mediate site specific cleavage at or near a restriction site (i.e., sequence of nucleotides recognized by the restriction enzyme) of double stranded nucleic acid molecules. Restriction endonucleases of interest include “rare cutting” restriction endonucleases, which recognize and mediate cleavage at specific DNA sequences of at least 8 bases pairs in length. Rare cutting restriction endonucleases include meganucleases, which typically have recognition sites comprising a double-stranded DNA sequence of 12 to 40 base pairs. Meganucleases include, without limitation, members of the LAGLIDADG family of homing endonucleases such as I-CreI and I-SceI. Rare cutting restriction endonucleases are discussed in Lamber et al. (1999) Mutat. Res. 433:159-68); Belford et al. (1997) Nucleic Acids Res 25:3379-88, and Jasin (1996) Trends Genet. 12:244-228. “Rare cutting” restriction endonucleases include I-Scel (Dujon (1989) Gene 92-119), which recognizes an 18 bp non-palindromic sequence, engineered I-Scel nucleases (Bibikova et al. (2001) Mol. Cell. Bio. 21:289-287, Niu et al. (2008) J. Mol. Biol. 382(1):188-202), I-CreI, which recognizes a 19-24 bp sequence, and engineered derivatives thereof (Arnould et al. (2011) Protein Eng. Des. Sel. 24(1-2):27-31, Durrenberger et al. (1993) Mol. Gen. Genet. 236(2-3):409-14), and the HO endonuclease of Saccharomyces cerevisiae (Haber (1995) Bioessays 17(7):609-20, NCBI accession number X90957). In some embodiments the restriction enzyme is an artificial restriction enzyme comprising a natural or engineered DNA-binding domain fused to a nuclease domain. Artificial restriction enzymes may include engineered FokI endonucleases, zinc finger nucleases, transcription activator-like effector nucleases, and artificial restriction enzymes generated using the Argonaute protein (PfAgo) from Pyrococcus furiosus.

“Sequence determination”, “sequencing” or “determining a nucleotide sequence” in reference to polynucleotides includes determination of partial as well as full sequence information of the polynucleotide. That is, the terms include sequences of subsets of the full set of four natural nucleotides, A, C, G and T, such as, for example, a sequence of just A's and C's of a target polynucleotide. That is, the terms include the determination of the identities, ordering, and locations of one, two, three or all of the four types of nucleotides within a target polynucleotide. In some embodiments, the terms include the determination of the identities, ordering, and locations of two, three or all of the four types of nucleotides within a target polynucleotide. In some embodiments sequence determination may be accomplished by identifying the ordering and locations of a single type of nucleotide, e.g. cytosines, within the target polynucleotide “catcgc . . . ” so that its sequence is represented as a binary code, e.g., “100101 . . . ” representing “c-(not c)(not c)c-(not c)-c . . . ” and the like. In some embodiments, the terms may also include subsequences of a target polynucleotide that serve as a fingerprint for the target polynucleotide; that is, subsequences that uniquely identify a target polynucleotide within a set of polynucleotides, e.g. all different RNA sequences expressed by a cell.

The terms “modification” or “alteration” as used herein in relation to a position or amino acid mean that the amino acid in the specific position has been modified compared to the amino acid of the wild-type protein.

Herein, the terms “peptide”, “polypeptide”, “protein”, “enzyme”, refer to a chain of amino acids linked by peptide bonds, regardless of the number of amino acids forming said chain.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41(GC) % (Marmur et al. (1962) J. Mol. Biol. 5:109-118).

### Scarless Template-Free Enzymatic Synthesis of Polynucleotides

Methods are provided for scarless template-free enzymatic synthesis of polynucleotides on a solid support. For enzymatic synthesis, an initiator is used, typically an oligonucleotide or polynucleotide at least three nucleotides in length, comprising a free 3′-hydroxyl group, which is extended using a template-free polymerase. The subject methods use an initiator that further comprises i) at least one cleavable group, which can be cleaved by a small molecule chemical agent, adjusting pH, heat, or photocleaved by illumination with light to release polynucleotides from a solid support after enzymatic synthesis is completed, and ii) at least one enzymatic cleavage site, which can be cleaved by a cleavage enzyme to remove polynucleotide scars.

Typically, enzymatic synthesis of polynucleotides takes place on substrates comprising a planar surface, such as, glass, silica, silicon oxide, plastic, or like surfaces, but it may also take place on other surfaces, such as, for example, non-planar surfaces (e.g., beads, particles), biological tissues, or surface-immobilized cDNAs extracted from tissues. The methods disclosed herein may include the use of an apparatus for highly parallel template-free enzymatic synthesis of polynucleotides, as described in International Application Publication No. WO 2020/020608 and WO 2022/013094; herein incorporated by reference in their entireties. In some embodiments, parallel synthesis is implemented by providing a support having discrete, non-overlapping, addressable sites where separate polynucleotides are synthesized and a means for controlling photoillumination, electrochemical conditions, or other reaction conditions at each site independently of the other sites. In some embodiments, such a parallel synthesis support is a planar support having a regular pattern of addressable sites, such as, a rectilinear pattern of sites, or a hexagonal pattern of sites. In some embodiments, the support is a planar support having an irregular pattern of addressable sites or complex pattern of addressable sites. In some embodiments, each site of a planar support is associated with one or more electrodes whose electrical characteristics may be controlled in an addressable manor independent of other electrodes of the planar support.

In some embodiments, the planar support comprises a plurality of sites comprising at least 256 sites, at least 512 sites, at least 1024 sites, at least 5000 sites, at least 10,000 sites, at least 25,000 sites, or at least 100,000 sites and as many as 10,000,000 sites or 300,000,000 sites. In some embodiments, the sites of the planar support is disposed in a regular array and each site is associated with at least one electrode integrated with the planar support. In some embodiments, the discrete site at which synthesis and/or sequencing take place each has an area in the range of from 0.25 mih² to 1000 mih², or from 1 mih² to 1000 mih², or from 10 mih² to 1000 mih², or from 100 mih² to 1000 mih². In some embodiments, the amount of a polynucleotide synthesized at each site is at least 10⁻⁶ fmol, or at least 10⁻³ fmol, or at least 1 fmol, or at least 1 pmol, or the amount of polynucleotide synthesized at each site is in the range of from 10⁻⁶ fmol to 1 fmol, or from 10⁻³ fmol to 1 fmol, or from 1 fmol to 1 pmol, or from 10⁻⁶ pmol to 10 pmol, or from 10⁻⁶ pmol to 1 pmol. In some embodiments, the number of polynucleotides synthesized at each site is in the range of from 1000 molecules to 10⁶ molecules, or from 1000 molecules to 10⁹ molecules, or from 1000 molecules to 10¹² molecules.

In some embodiments, enzymatically synthesized polynucleotides at each reaction site have lengths in the range of from 50 to 500 nucleotides. In some embodiments, enzymatically synthesized polynucleotides have lengths in the range of from 50 to 1000 nucleotides, including any length within this range.

In some embodiments, a plurality of polynucleotides are synthesized in parallel at discrete reaction sites addressable either for applying illumination (e.g., for cleaving a photocleavable group or deprotecting a 3′-O-photocleavable protecting group from an extension product) or electrode voltage (e.g., for deprotecting a 3′-O-electrochemically labile protecting group from an extension product). In some embodiments, the electrodes are arranged in an addressable electrode array in which individual electrodes may be controlled to generate a predetermined voltage difference between any given working electrode of the array and a counter electrode. In some embodiments, an array is provided comprising a plurality of reaction sites, wherein each reaction site has an initiator or an elongated fragment with a 3′-hydroxyl group protected with a 3′-O-electrochemically labile protecting group or a 3′-O-photocleavable protecting group.

To initiate a synthesis cycle, the 3′-hydroxyl groups of initiators or extension products thereof at selected sites are deprotected, for example, using a photoinduced or electrochemical deprotection method that is restricted to the location of the selected reaction sites. As described more fully below, in some embodiments, such localized deprotection may be effected by selective illumination or localized changes in voltage differences using site-specific electrodes. An exemplary synthesis could be carried out as follows: to the selectively deprotected sites is added a reagent comprising 3′-O-protected-dATPs and a template-free polymerase, such as a TdT, which is delivered to the deprotected sites. As described briefly below, the synthesis reagents may be delivered in a variety of ways, such as, by simple bulk flow over the solid support, droplets delivered by an inkjet device to individual reaction sites, or the like. After a predetermined time for the coupling reaction to advance to completion or to a suitable extent, the solid support is washed and the next group of polynucleotides (those for which C is the next monomer) at selected sites have their 3′-hydroxyl groups deprotected. To the selectively deprotected sites is added a reagent comprising 3′-O-protected-dCTPs and a template-free polymerase, such as a TdT, is delivered to the deprotected sites. Similar steps are performed for dGTPs and dTTPs, until the cycle is completed. The cycles are repeated until synthesis of the polynucleotides is completed.

Release of scarless polynucleotides from the surface of the solid support is achieved using a double cleavage method. Enzymatic synthesis is performed with an initiator comprising at least one cleavable group and at least one enzymatic cleavage site, which is positioned between the cleavable group(s) and the free 3′-hydroxyl group of the initiator that is extended by a template-free polymerase. After enzymatic synthesis of the polynucleotides is completed, the cleavable group(s) is cleaved to release polynucleotides from the solid support, and one or more cleavage enzymes are used to selectively cleave the polynucleotides at one or more enzymatic cleavage sites to produce scarless polynucleotides.

Various cleavable linkages or cleavable nucleotides may be used in the subject methods. As used herein, the term “cleavable site” or “cleavage site” refers to a nucleotide or backbone linkage of a single stranded nucleic acid sequence that can be excised or cleaved under predetermined conditions, thereby separating the single stranded nucleic acid sequence into two parts.

In certain embodiments, cleaving the cleavable group comprises exposing the cleavable group to heat, light, a chemical, or a pH change. In certain embodiments, the initiator comprises a cleavable group (i.e., cleavage site) that is chemically cleavable by a small molecule chemical agent.

In some embodiments, the chemical agent is delivered to the reaction sites using an inkjet or by bulk flow over the solid support.

In some embodiments, a cleavage site comprises a disulfide bond, which can be cleaved with a reducing agent such as, but not limited to, dithiothreitol (DTT) or tris (2-carboxyethyl) phosphine (TCEP).

In some embodiments, the cleavable group is a periodate-sensitive group such as, but not limited to, a diol, a 1,2-hydroxy ketone, a 1,2-diketone, an α-keto acid, an α-hydroxy acid, an amino acid, a 1,2-amino alcohol, or a 1,2-diamine, which can be cleaved with periodate.

In some embodiments, the cleavable group is an alkali-labile group, which can be cleaved with a base. In some embodiments, the alkali-labile group is a ribonucleotide (e.g., rU).

In certain embodiments, the cleavable group comprises a pH-sensitive chemical bond. For example, the cleavable group comprising the pH-sensitive chemical bond may include, without limitation, an imine, a hydrazone, an oxime, an amide, or an acetal. In some embodiments, cleaving the pH-sensitive chemical bond comprises generating a local change in pH around the cleavable group using an electrode, wherein the pH-sensitive chemical bond is cleaved when the pH is changed to a predetermined value. In some embodiments, each reaction site is operationally associated with an electrode. In some embodiments, one or more selected electrodes are used for cleaving the pH-sensitive chemical bond at one or more selected reaction sites at predetermined addresses on the surface. In other embodiments, cleaving the pH-sensitive chemical bond comprises delivering an acid or base to the reaction sites using an inkjet or by bulk flow over the solid support.

In other embodiments, the initiator comprises a cleavable group that is photocleavable and is cleaved by illuminating the surface with light from a light source. Exemplary photocleavable groups include, without limitation, arylcarbonylmethyl groups such as a phenacyl group, an o-alkylphenacyl group, a p-hydroxyphenacyl group, a p-hydroxyacetophenone, a 2,5-dimethylphenacyl group, and a benzoin group; nitroaryl groups such as an o-nitrobenzyl group, a nitrophenethyl group, an o-nitro-2-phenethyloxycarbonyl group, a nitroveratryl group, and an o-nitroanilide group; a coumarin-4-ylmethyl group, an arylmethyl group, an o-hydroxyarylmethyl group, a 2-(2-hydroxyalkyl)phenyl]ethenone group, a pivaloyl group; an arylsulfonyl group, a sisyl group, an α,β-unsaturated anilide group; a 2-pyrrolidino-1,4-benzoquinone group, a 5-(ethylen-2-yl)-1,4-naphthoquinone group, a 4-oxo-4-o-tolylbutanoate group, a triazine group, a 2-(2-isopropylbenzoyl)benzoate ester group, an arylmethyleneimino group, an xanthene group, and a pyronin group. For a description of photocleavable groups, see, e.g., Klan et al. (2013) Chem. Rev. 113:119-191; herein incorporated by reference. Photocleavable linkers for use with oligonucleotides are commercially available, for example, from Biosearch Technologies (Hoddesdon, United Kingdom). See, e.g., shop.biosearchtech.com/support/nac/photocleavable-modifiers-use-with-oligonucleotides.

In other embodiments, the initiator has no thymidine bases, no adjacent thymidine bases, or sufficiently few thymidine bases to avoid photocrosslinking of thymidine bases from said illuminating.

Any suitable method may be used to illuminate the surface of the solid support to photocleave the photocleavable group to release a polynucleotide from the surface after synthesis is completed. In certain embodiments, the surface is illuminated with a light source such as, but not limited to, a light-emitting diode (LED), a laser diode, or a lamp that emits light at a suitable wavelength to cleave the photocleavable group. The wavelength range of the light needed will depend on the type of photocleavable group that is used. In some cases, the light is ultraviolet (UV) light, visible light, or infrared (IR) light.

The entire surface may be illuminated to release polynucleotides at multiple reaction sites simultaneously. Alternatively, the surface can be selectively illuminated to release polynucleotides from the surface at selected reaction sites. In some embodiments, the surface is selectively illuminated by passing light from a light source through a photomask or with a digital micromirror device or a light-emitting diode array configured to selectively illuminate the surface at selected positions.

A photomask (also referred to as a “mask” herein) may be positioned between a light source and the surface of the solid support on which nucleic acid synthesis is performed to provide site-specific photoillumination of the surface, e.g., for cleavage of the photocleavable group. In some embodiments, the photomask comprises a plurality of regions comprising a light shielding opaque material (i.e., to prevent light from reaching the surface to block photocleavage at selected positions) and a plurality of regions comprising a light transmitting transparent material (i.e., to allow light to reach the surface to cause photocleavage at selected positions).

Photomasks can be designed to generate a light pattern based on the positions of reaction sites on the surface of the solid support. For example, photomasks can be designed to selectively transmit light to cleave a photocleavable protecting group to deprotect a 3′-O-protected extension product at selected reaction sites or cleave a photocleavable group to release polynucleotides from the solid support at selected reaction sites after synthesis is completed. In some cases, selective illumination of the surface with a series of photomasks may be used for photocleavage to selectively release polynucleotides from the solid support in a specific order for subpooling.

Photomasks can be generated by any suitable method known in the art. The photomask may include a pattern of an opaque material on the surface of a transparent substrate. For example, photomasks can be generated from transparent fused silica plates or a quartz substrate patterned with photoresist using lithography techniques. Photomasks can be designed to provide spatial illumination patterns corresponding to any configuration of reaction sites or electrodes on the surface of the solid support. In some embodiments, the illumination pattern is a grid pattern (e.g., reaction sites or electrodes organized in an array) or a more complex pattern corresponding to a more complex arrangement of reaction sites or electrodes on the surface. For a description of methods of manufacturing photomasks, see, e.g., Handbook of Photomask Manufacturing Technology (edited by Rizvi, CRC Press, 2005); Eynon and Wu Photomask Fabrication Technology (Professional Engineering, McGraw Hill, 2005); herein incorporated by reference in their entireties.

Alternatively, a digital micromirror device (DMD) can be used for selective spatial illumination of the surface of a solid support. A DMD is an optical micro-electrical-mechanical system (MEMS), which uses micromirrors to modulate light reflected off a mirror array. The micromirrors are adjusted to control the intensity and direction of light. A DMD can be used to project specified light patterns on a surface with precision. Digital micromirror devices are commercially available, for example, from Texas Instruments (Dallas, TX) and Atomica Corporation (Goleta, CA). The DMD can be configured to selectively project light onto the surface to selectively cleave a photocleavable protecting group to deprotect a 3′-O-protected extension product at selected reaction sites or cleave a photocleavable group to release polynucleotides from the solid support at selected reaction sites after synthesis is completed. In some cases, a DMD can be used to selectively illuminate different regions of the surface in a specified order to selectively release polynucleotides from the solid support in a specific order for subpooling.

A light-emitting diode array may also be configured to selectively illuminate the surface of a solid support at selected positions (see, e.g., International Patent Application Publication No. WO/2022/010934; herein incorporated by reference in its entirety). The light-emitting diode array can be configured to selectively illuminate the surface to selectively cleave a photocleavable protecting group to deprotect a 3′-O-protected extension product at selected reaction sites or cleave a photocleavable group to release polynucleotides from the solid support at selected reaction sites after synthesis is completed. In some cases, a light-emitting diode array can be used to selectively illuminate different regions of the surface in a specified order to selectively release polynucleotides from the solid support in a specific order for subpooling.

In some embodiments, the photocleavable group is an ortho-nitrobenzyl photocleavable linker. Methods of synthesizing and cleaving nucleic acids containing photocleavable linkers are described, for example, in U.S. Pat. No. 5,700,642., Venkatesan et al., J. Org. Chem. 61:525-529 (1996), Kahl et al, J. Org. Chem. 64:507-510 (1999), Kahl et al, J. Org. Chem. 63:4870-4871 (1998), Greenberg et al., J. Org. Chem. 59:746-753 (1994), Holmes et al., J. Org. Chem. 62:2370-2380 (1997), Nadji et al., J. Am. Chem. 114:9266-9269 (1992), and U.S. Pat. No. 5,739,386. Ortho-nitrobenzyl-based linkers, such as hydroxymethyl, hydroxyethyl, and Fmoc-aminoethyl carboxylic acid linkers, may be obtained commercially.

In certain embodiments, the photocleavage step is performed in an inert gas environment, or in a gas environment with reduced oxygen content, by comparison to air. As demonstrated in Example 3, the inventors have indeed shown that substitutions at the base level on the product oligonucleotide can be significantly reduced when performing the photocleavage step in an atmosphere with reduced oxygen content. In specific embodiments, the photocleavage step is performed with the solid support immersed in an inert gas environment, preferably in an environment comprising mostly argon or in an environment comprising mostly nitrogen.

In certain embodiments, each initiator further comprises a 5′-linker, wherein the 5′-linker is attached to the surface of the solid support. In some embodiments, the 5′-linker comprises a modification at the 5′-end to facilitate attachment to the solid support. For example, the 5′-linker may comprise an amino group (e.g., for attachment to an aldehyde or ketone-functionalized surface), biotin or desthiobiotin (e.g., for binding to a streptavidin functionalized surface), an azide (e.g., for attachment using click chemistry to an alkyne functionalized surface), an alkyne (e.g., 5′ hexynyl or 5-octadiynyl dU for attachment using click chemistry to an azide functionalized surface), Acrydite™ (e.g., for attachment to a thiol-functionalized surface), or digoxigenin (e.g., for attachment to a surface functionalized with anti-digoxigenin antibodies).

In certain embodiments, the 5′-linker further comprises a spacer (e.g., between the 5′-modification and the 5′-end of the 5′-linker). The spacer may be hydrophilic or hydrophobic. Exemplary spacers include, without limitation, a propyl spacer, a propanediol spacer, an ethylene glycol spacer, a triethylene glycol spacer, a triethylene glycol ethylamine spacer, a hexa-ethylene glycol spacer, a hexanediol spacer, a hexadecyl spacer, a polyethylene glycol spacer, and the like. In some embodiments, the nucleotide spacer is at least 5 atoms in length, at least 7 atoms in length, at least 10 atoms in length, at least 15, atoms in length, or at least 20 atoms in length, or longer. In some embodiments, the spacer is 2 atoms to 20 atoms in length, including any length in this range.

In certain embodiments, the initiator comprises a nucleotide spacer between the 5′-linker and the cleavable group(s). In some embodiments, the nucleotide spacer is at least 1 nucleotide in length, at least 5 nucleotides in length, at least 10 nucleotides in length, at least 15, nucleotides in length, or at least 20 nucleotides in length, or longer. In some embodiments, the nucleotide spacer is 10 to 20 nucleotides in length, including any length within this range.

In certain embodiments, the 5′-linker comprises a nucleotide sequence capable of hybridizing to a complementary nucleotide sequence of a probe attached to the surface of the solid support, wherein hybridization of the nucleotide sequence of the 5′-linker and the complementary nucleotide sequence of the probe immobilizes the initiator on the solid support, and wherein dehybridization of the nucleotide sequence of the 5′-linker and the complementary nucleotide sequence of the probe releases the initiator or extension products thereof from the solid support.

Selection of appropriate hybridization conditions is within the skill in the art (see, for example, Nucleic Acid Hybridization: A Practical Approach, editors B. D. Hames and S. J. Higgins, (1985) Oxford; Washington, DC; IRL Press). With respect to the stringency of conditions for hybridization, it is well known in the art that numerous equivalent conditions can be employed to establish a particular stringency by varying, for example, the following factors: the length and nature of probe and target sequences, base composition of sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. Dehybridization (i.e., dissociation of the hydrogen bonding between base pairs) can be accomplished, for example, by heating above the melting temperature or by acid or alkali treatment, and the like. The selection of hybridization and dehybridization conditions is well known (see, e.g., Green and Sambrook, Molecular Cloning: A Laboratory Manual, Cold Spring Harbor Laboratory Press, 4^{th} edition, 2012).

### Enzymatic Cleavage

The polynucleotides are enzymatically cleaved by one or more cleavage enzymes that selectively cleave the polynucleotides at one or more enzymatic cleavage sites to produce scarless polynucleotides (i.e., only containing a desired sequence of interest with no extraneous sequences). Enzymatic cleavage may be performed simultaneously with or after cleaving the cleavable group(s). Once the polynucleotides are released from the surface, the cleavage enzymes can more readily access the enzymatic cleavage sites to remove any polynucleotide scars such as those introduced by the initiator, the cleavable group(s), or cleavage products.

In some embodiments, the cleavage enzymes are selected from an endonuclease, a DNA glycosylase, and an exonuclease.

In some embodiments, an enzymatic cleavage site comprises an inosine or a deoxyinosine, which can be cleaved by a eukaryotic or a prokaryotic endonuclease V enzyme, respectively. In some embodiments, an Escherichia coli endonuclease V is used for cleavage of an enzymatic cleavage site comprising a deoxyinosine. The E. coli endonuclease V cleaves the second DNA phosphodiester bond 3′ to the deoxyinosine. Enzymes with endonuclease V activity are available commercially, for example, from New England Biolabs (Ipswich, MA) and NzyTech (Lisbon, Portugal). Typical cleavage times range from 5 to 60 minutes, or from 10 to 30 minutes. Optionally, endonuclease activity of the above enzymes may be heat inactivated by incubation at 65° C. or higher for 20 minutes. An endonuclease V comprising a His tag is available from NzyTech, which allows convenient removal of the enzyme from reaction mixtures in preparation of final products.

In other embodiments, an enzymatic cleavage site comprises a deoxyuridine. A uracil-DNA-glycosylase can be used to cleave the N-glycosidic bond to eliminate the uracil leaving an apyrimidinic site. The ribose at the apyrimidinic site, because of missing the glycosidic bond, is unstable and converts into a reactive open-chain aldehyde, hemiacetal, or its hydrate form. This unstable apyrimidinic site can be cleaved chemically or by an endonuclease having lyase activity. For example, the apyrimidinic site can be cleaved under basic or acidic conditions, which cause a β-elimination reaction, resulting in the formation of an α,β-unsaturated aldehyde and cleavage of the phosphodiester backbone 3′ to the apyrimidinic site. A further 6-elimination reaction results in strand cleavage 5′ to the apyrimidinic site. Alternatively, various endonucleases with lyase activity can be used to cleave the phosphodiester backbone at the apyrimidinic site created by the uracil-DNA glycosylase, including apurinic/apyrimidinic (AP) endonucleases and endonuclease VIII. Uracil-Specific Excision Reagent (USER), which is commercially available from New England Biolabs (Ipswich, MA), contains a mixture of uracil-DNA-glycosylase and endonuclease VIII in a single solution that can be used to cleave enzymatic cleavage sites comprising deoxyuridine. The endonuclease VIII cleaves the phosphodiester backbone on both the 3′ and 5′ sides of the apyrimidinic site.

In other embodiments, an enzymatic cleavage site comprises a restriction site that can be cleaved by a restriction endonuclease. The restriction endonuclease is preferably a “rare cutting” restriction endonuclease, which recognizes and mediates cleavage at or near a specific DNA sequence of at least 8 bases pairs in length. Rare cutting restriction endonucleases include meganucleases, which typically have recognition sites comprising a double-stranded DNA sequence of 12 to 40 base pairs. Meganucleases include, without limitation, members of the LAGLIDADG family of homing endonucleases such as I-CreI and I-SceI. Rare cutting restriction endonucleases are discussed in Lamber et al. (1999) Mutat. Res. 433:159-68); Belford et al. (1997) Nucleic Acids Res 25:3379-88, and Jasin (1996) Trends Genet. 12:244-228. “Rare cutting” restriction endonucleases include I-Scel (Dujon (1989) Gene 92-119), which recognizes an 18 bp non-palindromic sequence, engineered I-Scel nucleases (Bibikova et al. (2001) Mol. Cell. Bio. 21:289-287, Niu et al. (2008) J. Mol. Biol. 382(1):188-202), I-CreI, which recognizes a 19-24 bp sequence, and engineered derivatives thereof (Arnould et al. (2011) Protein Eng. Des. Sel. 24(1-2):27-31, Durrenberger et al. (1993) Mol. Gen. Genet. 236(2-3):409-14), and the HO endonuclease of Saccharomyces cerevisiae (Haber (1995) Bioessays 17(7):609-20, NCBI accession number X90957). In some embodiments, the restriction enzyme is an artificial restriction enzyme comprising a natural or engineered DNA-binding domain fused to a nuclease domain. Artificial restriction enzymes may include engineered FokI endonucleases, zinc finger nucleases, transcription activator-like effector nucleases, and artificial restriction enzymes generated using the Argonaute protein (PfAgo) from Pyrococcus furiosus. For a description of various natural and artificial rare cutting restriction endonucleases, see, e.g., Gunn et al. (2012) Methods Mol. Biol. 920:379-391; Lamber et al. (1999) Mutat. Res. 433:159-168; Belford et al. (1997) Nucleic Acids Res. 25:3379-88) and Jasin (1996) Trends Genet. 12:244-228, Katada et al. (2009) Chembiochem. 10(8):1279-88, Kim et al. (1996) Proc. Natl. Acad. Sci. USA. 93 (3):1156-1160, Maeder et al. (2008) Mol. Cell. 31 (2):294-301, Boch et al. (2011) Nature Biotechnology 29 (2):135-136, Juillerat et al. (2015) Scientific Reports 5:8150, Stoddard et al. (2006) Quarterly Reviews of Biophysics. 38 (1):49-95, Enghiad et al. (2017) ACS Synth. Biol. 6(5):752-757; herein incorporated by reference.

In some embodiments, the one or more cleavage enzymes are delivered to the reaction sites using an inkjet or by bulk flow over the solid support.

In certain embodiments, a cleavage enzyme is deactivated or removed after the enzymatic cleavage reaction to remove polynucleotide scars is completed. For example, a thermolabile cleavage enzyme may be used, wherein the cleavage enzyme can be deactivated with heat after performing the enzymatic cleavage reaction. Alternatively or additionally, a cleavage enzyme may further comprise an affinity tag to allow the cleavage enzyme to be readily removed from the synthesized polynucleotides Exemplary affinity tags include, without limitation, a polyhistidine tag, a glutathione-S-transferase tag, a thioredoxin tag, a chitin binding protein (CBP), a maltose binding protein (MBP) tag, a Strep-tag, a FLAG-tag, a HAT-tag, a NusA tag, a TAP-tag, an S-tag, an SBP-tag, an Arg-tag, a calmodulin-binding peptide tag, a cellulose-binding domain tag, a DsbA tag, a c-myc tag, and an epitope tag. In some embodiments, the cleavage enzyme is separated from the synthesized polynucleotides released from the surface by affinity purification using an affinity reagent that binds to the affinity tag. In some embodiments, the affinity reagent is attached to magnetic beads to allow magnetic separation techniques to be used to separate the cleavage enzyme from the synthesized polynucleotides.

In some embodiments, a cleavage enzyme further comprises at least one catchable group, which allows the cleavage enzyme to be separated from the scarless polynucleotides by contacting the catchable group with a capture agent that selectively binds to the catchable group. Catchable groups are described in greater detail further below.

In certain embodiments, an initiator further comprises one or more catchable groups, wherein the catchable groups are positioned between the cleavable group(s) and the enzymatic cleavage site(s) such that the polynucleotide scar segment removed from the plurality of polynucleotides by enzymatic cleavage retains the one or more catchable groups. Inclusion of a catchable group allows the polynucleotide scar segment to be readily removed from the scarless polynucleotides after the enzymatic cleavage reaction by contacting the catchable group with a capture agent that selectively binds to the catchable group and captures the polynucleotide scar segment.

By “capture” is meant that the polynucleotide scar segment and/or cleavage enzyme can be separated from the scarless polynucleotides and other components in a sample by virtue of the binding of the capture agent to the catchable group. One or more capture agents can be used in order to capture the polynucleotide scar segments and/or cleavage enzymes. Typically, a capture agent is associated with a solid support, either directly or indirectly. The solid support may include without limitation, non-magnetic or magnetic beads or particles, a resin, a membrane, the surface of a well, slide, or plate, or the surface of a channel or chamber of a microfluidic device.

A catchable group may be a ligand capable of functioning as a member of a specific binding pair. In these embodiments, the initiator comprises one member of the binding pair and the capture agent comprises the second member of the binding pair. Examples of binding pairs include, without limitation, biotin-streptavidin/avidin, polynucleotide-probe, antigen-antibody, DNA-binding protein-polynucleotide with target binding site, hormone-receptor, antagonist/agonist-receptor, aptamer-ligand, and lectin-carbohydrate binding pairs. The capture agent may be attached to a solid support such that when the polynucleotide scar segment and/or cleavage enzyme is contacted with the solid support, the polynucleotide scar segment and/or cleavage enzyme is removed from solution by its catchable group binding to the capture agent attached to the solid support. In some embodiments, the capture agent is attached to magnetic beads or magnetic particles to allow magnetic separation techniques to be used to remove the polynucleotide scar segment and/or cleavage enzyme from a mixture containing the scarless polynucleotides after enzymatic cleavage.

In some embodiments, the catchable group is biotin or desthiobiotin, and the capture agent is a biotin-binding protein (e.g., streptavidin or avidin). A solid support comprising a biotin-binding protein such as avidin or streptavidin as a capture agent may be used to remove the polynucleotide scar segments and/or cleavage enzymes. Biotinylated nucleotides are commercially available, for example, from Thermo Fisher Scientific (Waltham, MA), Jena Bioscience (Jena, Germany), and Biotium (Fremont, CA). Streptavidin-coated magnetic beads are commercially available, for example, from PerkinElmer (Waltham, Massachusetts), Thermo Fisher Scientific (Waltham, MA), New England Biolabs (Ipswich, MA), Sigma-Aldrich (St. Louis, MO), Spherotech (Lake Forest, IL), and ACROBiosystems (Newark, DE).

In some embodiments, the catchable group is digoxigenin, and the capture agent is an anti-digoxigenin antibody. A solid support comprising an anti-digoxigenin antibody as a capture agent may be used to remove the polynucleotide scar segments and/or cleavage enzymes. Nucleotides labeled with digoxigenin are commercially available, for example, from Jena Bioscience (Jena, Germany), Sigma-Aldrich (St. Louis, MO), and Enzo Life Sciences Inc. (Farmingdale, NY). Anti-digoxigenin antibody coated magnetic particles are available, for example, from Spherotech, Inc. (Green Oaks, Illinois) and PerkinElmer (Waltham, Massachusetts).

In some embodiments, the catchable group and the capture agent are click chemistry reaction partners. A solid support comprising a first click chemistry reaction partner as a capture agent and a catchable group comprising a second click chemistry reaction partner may be used to remove the polynucleotide scar segments and/or cleavage enzymes. In certain embodiments, the catchable group comprises an azide group, and the capture agent comprises an alkyne group that reacts with the azide group in a strain-promoted alkyne-azide cycloaddition (SPAAC) click chemistry reaction or a copper(I)-catalyzed azide-alkyne cycloaddition (CuAAC) click chemistry reaction. In certain embodiments, the catchable group comprises an alkyne group, and the capture agent comprises an azide group that reacts with the alkyne group in a strain-promoted alkyne-azide cycloaddition (SPAAC) click chemistry reaction or a copper(I)-catalyzed azide-alkyne cycloaddition (CuAAC) click chemistry reaction. In certain embodiments, the catchable group comprises a diaryl-strained-cyclooctyne, and the capture agent comprises a 1,3-nitrone that reacts with the diaryl-strained-cyclooctyne in a strain-promoted alkyne-nitrone cycloaddition (SPANC) click chemistry reaction. In certain embodiments, the catchable group comprises a 1,3-nitrone, and the capture agent comprises a diaryl-strained-cyclooctyne that reacts with the 1,3-nitrone in a strain-promoted alkyne-nitrone cycloaddition (SPANC) click chemistry reaction. In certain embodiments, the catchable group comprises a thiol, and the capture agent comprises an alkene that reacts with the thiol in a thiol-ene reaction. In certain embodiments, the thiol-ene reaction is a light-initiated, heat-initiated, or radical initiated thiol-ene reaction. For a description of click chemistry, see, e.g., Chen et al. Click Chemistry: Approaches, Applications, and Challenges (Nova Science Publishers, Inc., 2017), Click Chemistry for Biotechnology and Materials Science (edited by Lahann, Wiley, 2009), Rostovtsev et al. (2002) Angewandte Chemie International Edition. 41(14): 2596-2599, Kolb et al. (2003) Drug Discov Today. 8 (24):1128-1137; Baskin et al. (2007) Proc Natl Acad Sci USA 104(43):16793-16797, Agard et al. (2006) ACS Chem. Biol. 1: 644-648, Codelli et al. (2008) J. Am. Chem. Soc. 130:11486-11493, Gordon et al. (2012) J. Am. Chem. Soc. 134:9199-9208, Jiang et al. (2015) Soft Matter 11(30):6029-6036, Jang et al. (2012) Bioconjug Chem. 23(11):2256-2261, Ornelas et al. (2010) J Am Chem Soc. 132(11):3923-3931; Grim et al. (2015) J. Control Release 219:95-106; Scanlan et al. (2014) Molecules 19(11):19137-151; Hoyle et al. (2010) Angew Chem. Int. Ed. Engl. 49(9):1540-1573; van Dijk et al. (2009) Bioconjug Chem. 20(11):2001-2016, Ning et al. (2010) Angew. Chem. Int. Ed. 49:3065, McKay et al. (2012) Org. Biomol. Chem. 10:3066, MacKenzie et al. (2014) Curr. Opin. Chem. Biol. 21: 81-88, MacKenzie et al. (2014) Can J Chem. 92 (4): 337-340; herein incorporated by reference in their entireties.

In some embodiments, the capture agent is a capture oligonucleotide comprising a nucleotide sequence complementary to a target nucleotide sequence present only in the polynucleotide scar segment and not in the scarless polynucleotide product. Such a capture oligonucleotide may be used to remove polynucleotide scar segments comprising the target nucleotide sequence from a mixture containing scarless polynucleotides that lack the target nucleotide sequence. The capture oligonucleotide may be attached to a solid support to facilitate removal of the polynucleotide scar segments from a mixture containing scarless polynucleotides and/or other components. In some cases, two or more capture oligonucleotides are used in order to capture the polynucleotide scar segments after enzymatic cleavage.

In some embodiments, the catchable group comprises a nucleotide target sequence and the capture agent is an aptamer or a DNA or RNA binding protein that selectively binds to the nucleotide target sequence. For example, the initiator may comprise a nucleotide target sequence positioned between the cleavable group(s) and the enzymatic cleavage site(s) such that the polynucleotide scar segment removed from the plurality of polynucleotides by enzymatic cleavage comprises the nucleotide target sequence. A solid support comprising an aptamer or a DNA or RNA binding protein that selectively binds to the nucleotide target sequence may be used to remove the polynucleotide scar segments.

In some embodiments, the catchable group is a ligand, and the capture agent comprises an aptamer, antibody, or receptor that binds to the ligand. A solid support comprising an aptamer, antibody, or receptor that selectively binds to the ligand may be used to remove the polynucleotide scar segments and/or cleavage enzymes.

In some embodiments, a capture agent is attached to a solid support such as, but not limited to, non-magnetic or magnetic beads or particles, a resin, a membrane, the surface of a well, slide, or plate, or the surface of a channel or chamber of a microfluidic device.

### Enzymatic Nucleic Acid Synthesis

Enzymatic nucleic acid synthesis uses an enzymatic catalyst to carry out the polymerization of nucleotides. Enzymatic DNA synthesis is generally performed with an enzyme that catalyzes the addition of nucleotides to the 3′ end of a DNA molecule. More specifically, the process employs, without being limited thereto, enzymes which make possible the creation of a phosphodiester bond between a 3′-OH group of a nucleic acid fragment in the course of synthesis and the 5′-OH group of the nucleotide to be added during the enzymatic addition stage.

In some embodiments, enzymatic nucleic acid synthesis is performed with an enzyme capable of catalyzing the polymerization of nucleotides independently of the presence of a complementary strand (i.e., template). Such enzymes are capable of synthesizing nucleic acids in the absence of any complementary strand. In some cases, enzymatic nucleic acid synthesis may be performed with an enzyme that has the ability to synthesize single stranded nucleic acid fragments. The addition of nucleotides is thus advantageously carried out by the enzymatic route, by means of enzymes capable of polymerizing nucleotides without the presence of a template strand.

In some embodiments, the enzyme chosen for use in enzymatic nucleic acid synthesis is a template-free polymerase selected from translesion DNA polymerases of type η or ζ, polynucleotide phosphorylases (PNPases), template-independent RNA polymerases, terminal transferases, template-independent DNA polymerases, reverse transcriptases, 9° N DNA polymerases, or terminal deoxynucleotidyl transferases (TdT). These enzymes are expressed by certain cells of living organisms and can be extracted from these cells or purified from recombinant cultures.

In some embodiments, an engineered terminal deoxynucleotidyl transferase is used to perform enzymatic nucleic acid synthesis. Various variants of terminal deoxynucleotidyl transferase have been developed for this purpose. See, e.g., U.S. Pat. Nos. 11,208,637; 10,752,887; 10,435,676; and U.S. Patent Application Publication No. 2022/0002687; herein incorporated by reference in their entireties. In some embodiments, an engineered reverse transcriptase is used to perform enzymatic nucleic acid synthesis. For example, human immunodeficiency virus type-1 and Moloney murine leukemia virus reverse transcriptases may be used. Engineered Moloney murine leukemia virus reverse transcriptase variants are commercially available such as the SuperScript IV reverse transcriptase from Thermo Fisher (Waltham, MA) and SMARTScribe reverse transcriptase from Clonetech (Mountain View, Calif.).

In some embodiments, an engineered 9° N DNA polymerase is used to perform enzymatic DNA synthesis. Engineered 9° N DNA polymerase variants are commercially available, including duplases from Centrillion Technology Holdings Corporation (Grand Cayman, KY) and the Therminator Thermococcus sp. DNA polymerase from New England Biolabs (Ipswich, MA). See also, e.g., Hoff et al. (2020) ACS Synth Biol 9(2):283-293; Gardner et al. (2019) Front. Mol. Biosci. 6:28; herein incorporated by reference in their entireties.

A cycle of the enzymatic synthesis process, leading to the addition of a nucleotide to a nucleic acid strand, comprises two successive steps, an elongation step and a deprotecting step respectively. During the elongation step, the polymerase adds a nucleotide comprising a protecting group to a nucleic acid strand. Then the protection group is removed from this newly added nucleotide, to be able to perform additional cycles.

Synthesis of a complete nucleic acid by template-free enzymatic nucleic acid synthesis typically comprises repeated cycles of steps, in which a selected nucleotide is coupled to an initiator or growing chain in each cycle. As used herein, an “initiator” (or equivalent terms, such as, “initiating fragment”, “initiator nucleic acid”, “initiator oligonucleotide”, “initiator polynucleotide”, or the like) refers to an oligonucleotide or polynucleotide comprising a free 3′-hydroxyl group, which can be further elongated by a template-free polymerase (e.g., TdT). In some cases, the initiator is photocleavable. In one embodiment, the initiator is a DNA initiating fragment. In an alternative embodiment, the initiator is an RNA initiating fragment. In some embodiments, an initiator comprises between 3 and 100 nucleotides. In some embodiments, an initiator comprises between 3 and 20 nucleotides. In some embodiments, the initiator is single-stranded. In alternative embodiments, the initiator is double-stranded. In some embodiments, an initiator may comprise a non-nucleic acid compound having a free hydroxyl group to which a TdT may couple a 3′-O-protected dNTP (see, e.g., Baiga, U.S. patent publications US2019/0078065 and US2019/0078126; herein incorporated by reference). The general elements of template-free enzymatic synthesis are described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999).

Initiators are provided, for example, attached to solid support, with free 3′-hydroxyl groups. To the initiator (or elongated initiator polynucleotides in subsequent cycles) are added a 3′-O-protected-dNTP and a template-free polymerase, such as a TdT or a variant thereof (e.g., Ybert et al, WO/2017/216472) under conditions effective for the enzymatic incorporation of the 3′-O-protected-dNTP onto the 3′-end of the initiator (or elongated initiator polynucleotides). This reaction produces elongated initiator polynucleotides whose 3′-hydroxyls are protected. If the elongated initiator polynucleotide contains a completed sequence, then the 3′-O-protection group may be removed, or deprotected, and the desired sequence may be cleaved from the original initiator. Such cleavage may be carried out using any of a variety of single strand cleavage techniques, for example, by inserting a cleavable nucleotide at a predetermined location within the original initiator. An exemplary cleavable nucleotide may be a uracil nucleotide which is cleaved by uracil DNA glycosylase or an inosine nucleotide which is cleaved by endonuclease V. If the elongated initiator polynucleotide does not contain a completed sequence, then the 3′-O-protection groups are removed to expose free 3′-hydroxyls and the elongated initiator polynucleotides are subjected to another cycle of nucleotide addition and deprotection.

In some embodiments, 3′-O-protection groups are electrochemically labile groups. That is, deprotection or cleavage of the protection group is accomplished by changing the electrochemical conditions in the vicinity of the protection group, which results in cleavage. Such changes in electrochemical conditions may be brought about by changing or applying a physical quantity, such as a voltage difference or light to activate auxiliary species which, in turn, cause changes in the electrochemical conditions at the site of the protection group, such as an increase or decrease in pH. In some embodiments, electrochemically labile groups include, for example, pH-sensitive protection groups that are cleaved whenever the pH is changed to a predetermined value. In other embodiments, electrochemically labile groups include protecting groups which are cleaved directly whenever reducing or oxidizing conditions are changed, for example, by increasing or decreasing a voltage difference at the site of the protection group.

In other embodiments, the 3′-O-protection groups are photoremovable groups. That is, deprotection or cleavage of the protection group is accomplished by illuminating the surface of the solid support in the vicinity of the protection group, which results in photocleavage. In non-illuminated areas on the surface, 3′-O-protected initiators and 3′-O-protected extension products retain the 3′-photoremovable protecting group and remain unextendible. As described above, the surface of the solid support can be selectively illuminated by passing light from a light source through a photomask or using a digital micromirror device or a light-emitting diode array configured to selectively illuminate the surface such that the 3′-photoremovable protecting group is removed at selected locations, for example, at selected reaction sites where nucleic acid synthesis is desired. If a photoremovable protecting group is used in combination with an initiator comprising a 5′-photocleavable group (e.g., for release of polynucleotides from the solid support after synthesis), the photoremovable protecting group and the photocleavable group should be selected such that they require different wavelengths for photocleavage.

Photoremovable protecting groups may include, without limitation, 2-nitrobenzyl, 4,5-dimethoxy-2-nitrobenzyl, 6-nitropiperonyl, and 9-anthrylmethyl functional groups. For a description of photoremovable protecting groups that may be used in synthesis, see, e.g., Matthews et al. (2016) Org. Biomol Chem. 14(35):8278-88, Matthews et al. (2017) Curr. Protoc. Nucleic Acid Chem. 71:13.17.1-13.17.38, Bohacova et al. (2018) Org. Biomol. Chem. 16(9):1527-1535, Klan et al. (2013) Chem. Rev. 113(1): 119-191; herein incorporated by reference. In some embodiments, photoillumination-induced deprotection may involve the generation of an acid locally to deprotect the initiator (see, e.g., Gao et al, U.S. Pat. Nos. 6,426,184, 7,491,680 and 7,838,466; herein incorporated by reference).

In some embodiments, an ordered sequence of nucleotides are coupled to an initiator using a template-free polymerase, such as TdT, in the presence of 3′-O-protected dNTPs at each synthesis step. In some embodiments, the method of synthesizing an oligonucleotide comprises the steps of (a) providing an initiator having a free 3′-hydroxyl; (b) reacting under extension conditions the initiator or an extension intermediate having a free 3′-hydroxyl with a template-free polymerase in the presence of a 3′-O-protected nucleoside triphosphate to produce a 3′-O-protected extension intermediate; (c) deprotecting the extension intermediate to produce an extension intermediate with a free 3′-hydroxyl group; and (d) repeating steps (b) and (c) until the polynucleotide is synthesized. (The terms “extension intermediate,” “extension product” and “elongation fragment” are used interchangeably). In some embodiments, an initiator is provided as an oligonucleotide attached to a solid support, e.g., by its 5′ end. The above method may also include washing steps after the reaction, or extension, step, as well as after the de-protecting step. For example, the step of reacting may include a sub-step of removing unincorporated nucleoside triphosphates, e.g., by washing, after a predetermined incubation period, or reaction time. Such predetermined incubation periods or reaction times may be a few seconds, e.g., 30 seconds, to several minutes, e.g., 30 minutes.

The 3′-O-blocked dNTPs employed may be purchased from commercial vendors or synthesized using published techniques (see, e.g., U.S. Pat. No. 7,057,026; Guo et al, Proc. Natl. Acad. Sci., 105(27): 9145-9150 (2008); Benner, U.S. Pat. No. 7,544,794; herein incorporated by reference in their entireties).

The above method may also include capping step(s) as well as washing steps after the reacting, or extending, step, as well as after the deprotecting step. As mentioned above, in some embodiments, capping steps may be included in which non-extended free 3′-hydroxyl groups are reacted with compounds that prevents any further extension of the capped strand. In some embodiments, the compound is a dideoxynucleoside triphosphate. In other embodiments, non-extended strands with free 3′-hydroxyl groups are degraded by treating them with a 3′-exonuclease activity, e.g., Exo I. For example, see Hyman, U.S. Pat. No. 5,436,143. Likewise, in some embodiments, strands that fail to be deblocked may be treated to either remove the strand or render it inert to further extensions.

In some embodiments, reaction conditions for an extension or elongation step may comprising the following: 2.0-50 M purified TdT, preferably around 25 M purified TdT; 125-600 M 3′-O-protected dNTP (e.g. 3′-O—NH₂-protected dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3 minutes.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. It should be understood, however, that the intention is not to limit the invention to the particular embodiments described. The intention is to cover all modifications, equivalents, and alternatives falling within the spirit and scope of the invention.

### Example 1: Nucleic Acid Synthesis with Double Cleavage Scarless Release

Endo V (dInosine) cleavage and USER (dUracil) cleavage have been tried on planar supports and achieved only modest levels of DNA release. This is believed to be due to planar supports having a high level of functional groups for covalent immobilization and a dense layer of DNA initiator that is harder for enzymes to access—the cleavage site is always closer to the surface than the site of synthesis and the longer the DNA gets, the bigger the barrier to entry becomes. Reducing DNA density is a solution but difficult and not favorable for applications where parallelization and mass are equally important.

Non-enzymatic means of DNA cleavage (e.g. orthbenzyl, periodate, alkaline, disulfide) rely on much smaller cleavage agents (small molecules or photons) and can be very efficient but the drawback is that they leave a scar. Polymerases used for enzymatic DNA synthesis cannot prime off them, and a polynucleotide sequence is needed at the distal end. Furthermore, being able to cleave efficiently without a scar in a spatial manner is also desirable, e.g., for sub-pooling.

Here, we use a double cleavage strategy involving initial release of the DNA from the surface using light or a small molecule chemical with near simultaneous or subsequent enzymatic cleavage for release of the scar. The chemical can be spatially deposited (e.g., by inkjet) or flowed over the surface. If illumination is used for photocleavage, the light can be directed in a non-spatial or spatial manner, e.g., using a photomask or digital micromirror device (DMD). If illumination is used, the initiator for enzymatic DNA synthesis preferably has minimal T bases to avoid photocrosslinking of thymidine bases (e.g., polyC) or is predominantly RNA rather than DNA. Preferably, photocleavage is performed in the enzyme buffer with or without the enzyme present. In some cases, the enzyme is added subsequently to photocleavage. Preferably, the photo/chemically cleaved scar and enzyme possess a biotin or another group that allows easy separation of the scar/enzyme from the synthesized strand. Preferably, there is more than 1 photo/chemical cleavable group. Release of the DNA photo/chemically allows the enzyme to work in a more favourable environment without steric hindrance to generate scar-free DNA. The enzyme is preferably neutralized before it or a nucleotide can transfer to another spot where there may be strands that have unblocked 3′-hydroxyl groups.

Photocleavage should be avoided if the DNA has poly-T regions to prevent photocrosslinking of thymidine bases. For synthesis of DNA with poly-T stretches in sequence, chemical cleavage is preferable. In some cases, an RNA initiator is used to avoid photocrosslinking of thymidine bases because RNA has a greater resistance to UV damage than DNA.

### Example 2: Optimization of Cleavage Strategy

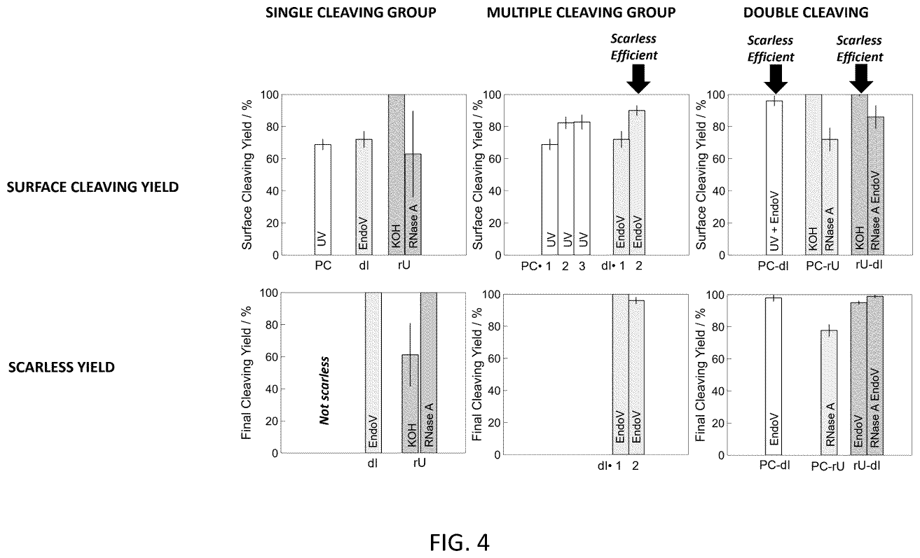

FIG. 4 shows cleaving yields with a single cleavable group, two cleavable groups, and multiple cleavable groups. We predicted that multiple groups of the same type would give higher surface cleavage yields from surfaces. The improvement would depend on how efficient a single cleavable group of a particular type is. If a given type is highly efficient (alkaline cleavage of rU), there will be proportionally less benefit of having 2 groups of this type and even less of having 3 groups. The best yields of the scarless oligo product were obtained using a double cleavage strategy. Preferably, the 2 cleavable groups are different and the group closest to the 3′ end is an enzyme cleavable group (e.g., dI or dU).

One can reasonably assume that combining more cleavable groups and double cleavage would give the best performance. We reasoned that anything that helps decrease density and makes the surface more accessible to the enzyme that cleaves scarlessly would improve perfomance. Scarless cleavage is optimal when the polynucleotide has already been released into solution.

Preferably, the DNA initiator has a spacer of 5, and preferably 10 to 20 nucleotides (nt) before the first cleavage site on the 5′ end (e.g., (surface)-(option spacer e.g., TEG or C12)-(10-20 nucleotide spacer)-(cleavage site(s) of type 1 dispersed in a nucleotide spacer)-(enzymatic cleavage site)-3′). In the case of dlnosine, the enzymatic cleavage site may be 1 nucleotide, i.e., dIT-3′.

The preferred combinations of those tested are PC-dI and rU-dI. It should be noted that rU-dI cleavage can be done via alkaline conditions or enzymatically using RNase. We prefer the latter because the high pH/high salt used for alkaline cleavage can create problems for the enzyme and necessitate dilution. If using alkaline conditions to remove the rU-dI or PC-dl efficiently from the surface then an alternative to dilution is to precipitate the cleaved DNA, and then use Endo V to cleave the dl. Alternatively, the rU-dI or PC-dI may be cleaved (at the rU position and surface and at the surface, respectively) with ammonium hydroxide. The ammonium hydroxide may then be evaporated off and the dI cleaved by addition of Endo V enzyme.

Other possibilities include chemically cleavable groups (e.g., periodate, TCEP) and dehybridization as a means of initial release, which may be more compatible with the scarless cleavage method than alkaline cleavage.

As regards the scarless cleavage method at the 3′-end, the options include dInosine (+Endo V), dUridine (+USER), and a restriction enzyme. For a review of scarless cleavage methods with endonuclease V and an initiator with a 3′-penultimate deoxyinosine, see e.g., U.S. Pat. No. 11,359,221; herein incorporated by reference in its entirety.

In a preferred embodiment, the scarless cleavage and the other cleavage method are performed simultaneously. However, cleavage can be performed as a 2-step process e.g., photocleavage in water or an enzyme buffer, and subsequently adding the remaining enzyme reagents (i.e., asynchronous). More than one type of cleavage group can be used for the interior cleavage (e.g., diol (periodate cleavage)+ortho-nitrobenzyl (photocleavage), or rU (cleaved by RNase)+ortho-nitrobenzyl (photocleavage)) i.e., in addition to the 3′ enzymatic cleavage. In some embodiments, cleavage of one or more sites or all sites is triggerable, e.g., by the action of an electrode. Cleavage agents can be applied in a spatial or bulk manner.

The enzyme used for scarless cleavage as well as the scar ideally have a catchable group so they can be removed from the product. Catching methods and catchable groups may include the use of copper and copper-free click chemistry (e.g., with azide and alkynes, or equivalents).

### Example 3: Optimization of Photocleavage Using an Inert Gas Environment

The present inventors have discovered that during photocleavage in ambient air, the synthesized oligonucleotides undergo a non-negligeable amount of substitutions, in particular G to T transversions. This example demonstrates that these substitutions can be significantly reduced when the photocleavage step is performed in an inert gas atmosphere, thereby demonstrating that the substitutions are at least in part due to oxidation during photocleavage.

Photocleaving was carried out at low oxygen pressure to attempt to minimize G>T transversion. Ambion nuclease-free water was degassed by bubbling argon for 30 min. An incubation chamber, equipped with the UV lamp, was flushed with Argon for 20 minutes prior photocleaving and kept under argon flow for all the duration of the photocleaving. In one single-well gasket filled with 1.4 mL of degassed Ambion nuclease-free water, illumination was carried out at 365 nm for 18 min. The cleaved oligonucleotides were dried by speedvac (~3 h min, 45° C.), re-suspended in 10 μL Ambion nuclease-free water (stirred and vortexed well), and submitted to NGS. The printed oligonucleotide sequences comprised partial P5/partial P7 primer sites, and their full sequences were as follows:

Full sequence e13 as printed, including the partial P5 and partial P7 primers:

(Seq ID No: 1)
CGACGCTCTTCCGATCTGAACTTCAACTTCAACGGCCTTACCGGCAC
CGGCGTGCTGACCGAGAGCAGGAGATCGGAAGAGCACACG.

Payload for sequence e13, without the partial P5 and partial P7 primers:

(Seq ID No: 2)
GAACTTCAACTTCAACGGCCTTACCGGCACCGGCGTGCTGACCGAGA
GCA.

Full sequence es as printed, including the partial P5 and partial P7 primers:

(Seq ID No: 3)
CGACGCTCTTCCGATCTATCCGACTGAAAGTACGCGAGATAGACTGC
TAGAAATCCTTGTGATTACGGGAGATCGGAAGAGCACACG.

Payload for sequence cs, without the partial P5 and partial P7 primers:

(Seq ID No: 4)
ATCCGACTGAAAGTACGCGAGATAGACTGCTAGAAATCCTTGTGATT
ACG.

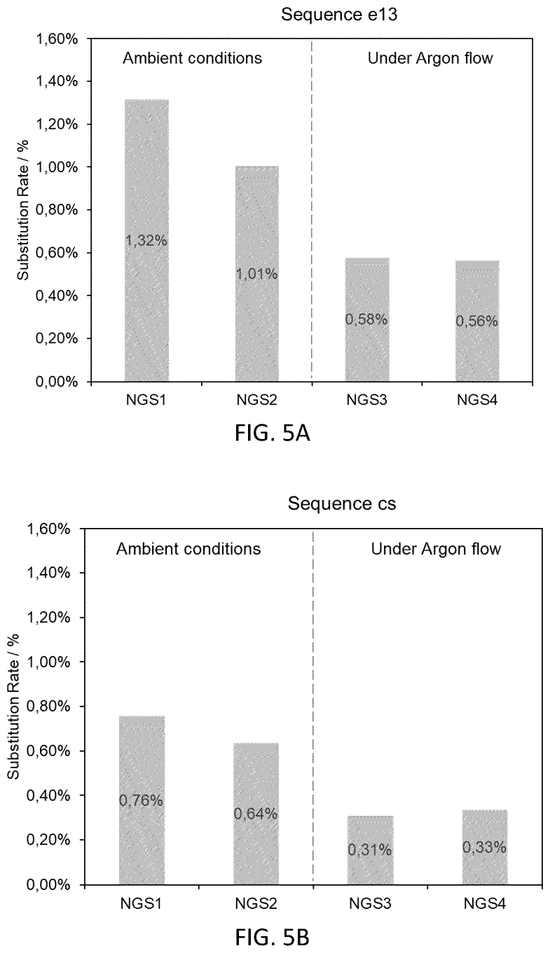

As can be seen in FIG. 5, the total substitution rate is significantly reduced when photocleaving is performed under argon flow (experiments labelled NGS3 and NGS4, two repeats) versus under ambient conditions (experiments labelled NGS1 and NGS2, two repeats), both for the e13 sequence (FIG. 5A) and for the cs sequence (FIG. 5B).

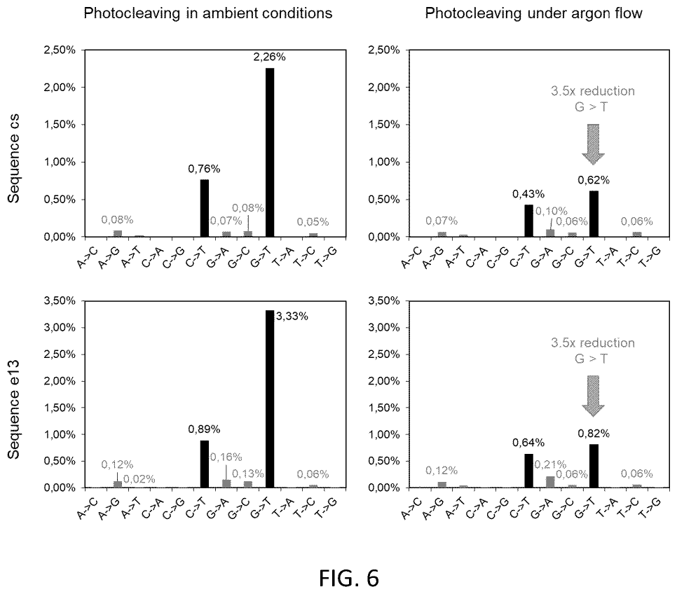

FIG. 6 provides details of the effect of the inert gas flow on a substitution type by substitution type basis, both for the cs sequence (above) and the e13 sequence (below). It is particularly noteworthy that the G to T transversion, the most prominent one under ambient conditions, is reduced by a factor of 3.5 under inert gas flow. It is to note that this reduction is not complete because the experimental conditions did not allow a perfect air-tight environment during photocleaving.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

1. A method of enzymatically synthesizing a plurality of polynucleotides, the method comprising:
(a) providing a plurality of reaction sites on a solid support;
(b) immobilizing a plurality of initiators on a surface of the solid support, wherein each initiator comprises a free 3′-hydroxyl group, at least one cleavable group, and at least one enzymatic cleavage site, wherein said at least one enzymatic cleavage site is positioned between the free 3′-hydroxyl group and said at least one cleavable group;
(c) contacting the surface of the solid support with a 3′-O-protected nucleoside triphosphate and a template-free polymerase under suitable conditions for enzymatic extension of the initiators, wherein incorporation of the 3′-O-protected nucleoside triphosphate results in production of a 3′-O-protected extension product at each of the reaction sites;
(d) performing one or more cycles of i) deprotecting the 3′-O-protected extension products at the reaction sites, wherein extension products having free 3′-hydroxyl groups are formed at the reaction sites, and ii) contacting the surface of the solid support with another 3′-O-protected nucleoside triphosphate and a template-free polymerase under suitable conditions for enzymatic extension of the extension products having free 3′-hydroxyl groups;
(e) repeating step (d) until synthesis of the plurality of polynucleotides is completed;
(f) cleaving said at least one cleavable group, thereby releasing the plurality of polynucleotides from the surface of the solid support; and
(g) enzymatically cleaving said at least one enzymatic cleavage site, thereby removing polynucleotide scars from the plurality of polynucleotides to produce a plurality of scarless polynucleotides, wherein said enzymatically cleaving the at least one enzymatic cleavage site is performed simultaneously with or after said cleaving the at least one cleavable group.

2. The method of claim 1, wherein the plurality of reaction sites is organized in a spatially addressable array on the solid support.

3. The method of claim 1, wherein said enzymatically cleaving comprises contacting the plurality of polynucleotides with one or more cleavage enzymes.

4. The method of claim 3, wherein the one or more cleavage enzymes are thermolabile, wherein after performing step (g), the method further comprises deactivating the one or more cleavage enzymes with heat.

5. The method of claim 3, wherein the one or more cleavage enzymes are selected from an endonuclease, a DNA glycosylase, and an exonuclease.

6. The method of claim 5, wherein said at least one enzymatic cleavage site comprises an inosine or a deoxyinosine, wherein said enzymatically cleaving comprises contacting the plurality of polynucleotides with an endonuclease V.

7. The method of claim 5, wherein said at least one cleavage site comprises a deoxyuridine, wherein said enzymatically cleaving comprises contacting the plurality of polynucleotides with an uracil-DNA-glycosylase and an endonuclease VIII or an apurinic/apyrimidinic (AP) endonuclease.

8. The method of claim 3, wherein the one or more cleavage enzymes further comprise one or more affinity tags.

9. The method of claim 3, wherein the one or more cleavage enzymes further comprise at least one catchable group, wherein after performing step (g), the method further comprises separating the one or more cleavage enzymes from the plurality of scarless polynucleotides by contacting said at least one catchable group with at least one capture agent that selectively binds to said at least one catchable group.

10. The method of claim 1, wherein said cleaving said at least one cleavable group comprises exposing said at least one cleavable group to heat, light, a chemical agent, or a pH change.

11. The method of claim 10, wherein said at least one cleavable group comprises a disulfide bond, and said cleaving comprises reacting the disulfide bond with a reducing agent.

12. The method of claim 10, wherein said at least one cleavable group comprises a pH-sensitive chemical bond.

13. The method of claim 12, wherein said cleaving the pH-sensitive chemical bond comprises generating a local change in pH around the cleavable group using an electrode, wherein the pH-sensitive chemical bond is cleaved when the pH is changed to a predetermined value.

14. The method of claim 10, wherein said at least one cleavable group is photocleavable, and said cleaving comprises illuminating the surface with light from a light source.

15. The method of claim 1, wherein each initiator further comprises a 5′-linker, wherein the 5′-linker is attached to the surface of the solid support.

16. The method of claim 8, wherein the one or more affinity tags are selected from a group consisting of a polyhistidine tag, a glutathione-S-transferase tag, a chitin binding protein (CBP), a maltose binding protein (MBP) tag, a Strep-tag, a FLAG-tag, and an epitope tag.

17. The method of claim 11, wherein the reducing agent is dithiothreitol (DTT) or tris (2-carboxyethyl) phosphine (TCEP).

18. The method of claim 14, wherein said cleaving is performed in a gas environment with reduced oxygen content.


SEQUENCE LISTING (4 sequences)

SEQ ID NO: 1 - DNA, 87 bp, synthetic construct
  source 1..87: mol_type = other DNA
cgacgctcttccgatctgaacttcaacttcaacggccttaccggcaccggcgtgctgaccgagagcaggagatcggaagagcacacg

SEQ ID NO: 2 - DNA, 50 bp, synthetic construct
  source 1..50: mol_type = other DNA
gaacttcaacttcaacggccttaccggcaccggcgtgctgaccgagagca

SEQ ID NO: 3 - DNA, 87 bp, synthetic construct
  source 1..87: mol_type = other DNA
cgacgctcttccgatctatccgactgaaagtacgcgagatagactgctagaaatccttgtgattacgggagatcggaagagcacacg

SEQ ID NO: 4 - DNA, 50 bp, synthetic construct
  source 1..50: mol_type = other DNA
atccgactgaaagtacgcgagatagactgctagaaatccttgtgattacg